(19) United States
(12) Patent Application Publication
(10) Pub. No.: US 2002/0109819 A1
(43) Pub. Date: Aug. 15, 2002

(54) Method and apparatus for low bandwidth transmission of data utilizing of the human eye anatomy

(75) Inventor: Miron Tuval, Ramat Gan (IL)

Correspondence Address: BROWDY AND NEIMARK, P.L.L.C., 624 NINTH STREET, NW, SUITE 300, WASHINGTON, DC, 20001-5303, US

(73) Assignee: TVEYE INC.

(21) Appl. No.: 10/075,385

(22) Filed: Feb. 15, 2002

Related U.S. Application Data
(60) Provisional application No. 60/268,989, filed on Feb. 15, 2001.

(51) Int. Cl. A61B 3/14

(52) U.S. Cl. 351/206

(57) ABSTRACT

A method for the generation of an image on a selectable part of the retina of a viewer's eye, to form an ocular image on the retina. The method includes the step of receiving an image being composed of a number of image elements that correspond to the number of cone photoreceptors of a viewer's fovea, so as to constitute a received image. The method further includes the step of displaying the received image so as to constitute a displayed image. The method further includes the step of projecting the displayed image onto a viewer's fovea area, so as to constitute a foveal image, such that a number of image elements of the foveal image corresponds to the number of cone photoreceptors of the viewer's fovea. The method further includes the step of projecting said displayed image onto a viewer's retina so as to constitute a retinal image.

### FIELD OF THE INVENTION

This invention relates to a method and system in the field of an efficient projection and display of images, taking advantage of the anatomy of the eye.

### BACKGROUND OF THE INVENTION

Two families of displays are known in the art: screens and virtual displays (such as holograms). Digital displays are composed of a number of image elements typically called pixels, which may be equally spaced all over the display surface. It is commonly accepted that the higher the density of the pixels, the better the quality of the display. Therefore certain displays exist which are characterized by having, say, hundreds of thousands pixels or more.

In a digital image, each pixel is typically represented separately by a characteristic number of bits, depending on the representation method. Thus, for example, in a video display of, say, 30 frames per second, where each frame is composed of 640 * 480 pixels (according to the VGA standard) and each pixel requires 24 bit (according to RGB), a very large volume of data is required, which inevitably entails a high bandwidth for transmission. Note that there are many known per se standards for video transmission and data compression. For instance, the H.320 standard requires only 26,000 pixels per video same.

In the field of personal displays, a heads up display (HUD hereinbelow) of a known design is typically a head-mounted display unit wherein the display unit comprises a screen on which an image is generated. Current HUD screens are fairly cumbersome and may cover much or all of the wearer's field of view. One example of a patent relating to HUD is U.S. Pat. No. 6,140,990 (Oct. 31, 2000, Spitzer, Gale and Jacobsen), which discloses a head-mounted display system including a high resolution active matrix display, which reduces center of gravity offset in a compact design. The active matrix display can be e.g. a liquid crystal display, a light emitting display etc.

With HUDs, as with other types of displays, the number of the required pixels for the generation of the image is a dominant factor in the computational and communication capacity requirements of the systems.

It is import to consider some key concepts in the anatomy (FIG. 1) and physiology of the eye, in order to understand the limitations of the existing display devices, including HUDs.

The cornea 110 is the transparent, dome-shaped refracting surface covering the front of the eye, which provides initial constant incoming light focusing. The light that impinges on the cornea is focused by it and enters the eyeball through the pupil 120. The extent of the pupil's aperture determines the amount of light entering the eye. The lens 130 focuses the light further onto the retina 140.

FIG. 2 shows an object 186 delimited by ABCD, subtended by a large spatial angle at the eye. The retinal image 182 delimited by A′B′C′D′ formed on: the retina 184 by object 186. As seen, retinal image 182 covers a large part of the retina 184.

The retina (FIG. 3) is a multi-layered sensory tissue that lines the back of the eyeball. At the back of the retina there is a layer 200 of photoreceptor cells (hereinbelow “photoreceptors”), which convert light energy into neural signals, sent to the visual cortex of the brain via the optic nerve 150 (FIG. 1). There are two types of photoreceptors in the retina: rod photoreceptors 210 (hereinbelow “rods”) and cone photoreceptors 220 (hereinbelow “cones”). The retina 140 comprises about 75-150 million rods 210, and 5-8 million cones 220. The rods 210 are responsible for dim light and gray-level vision, as they distinguish between light intensities only. In bright light the cones 220, which are less sensitive to light but distinguish between colors, provide color vision.

In FIG. 1, the macula lutea 160 (hereinbelow “macula”) is located slightly off the retinal optical center, temporal to the optic nerve. It is a small and highly sensitive area of the retina responsible for detailed central vision. The, fovea centralis 170 (hereinbelow “fovea”) is the center of the macula. The fovea 170 comprises 75,000-200,000 densely packed cones and a considerably smaller number of rods or none at all. The macula 160 includes cones, though at a lower density than the foveal cone density, as well as rods. The retinal cone density drops sharply as the distance from the fovea increases, and in most of the retinal surface, most of the photosensitive organs are rods.

FIG. 4 schematically represents the cone density gradient in the retina, showing the high cone density 250 in the fovea, the lower cone density 251 in the macula and the still lower density 252 in the other parts of the retina. Note that for illustrative purposes only, the cones are depicted in the drawings in a rectangular shape.

Neural signals generated by the photoreceptors are conducted to the brain by the optic nerve 150 that is composed of about 1 million neural fibers. Most of the fibers are connected to more than one photoreceptor. However, the foveal cones are each connected to one fiber in the optic nerve 150. Therefore, the stimulation of the fovea 170 is required to produce the best, most accurate and acute vision in normal daylight conditions. When the eye is directed at an object, the part of the image that is focused on the fovea 170 is seen most sharply. The acuity decreases when the image falls on the macula 160 and is lower still in the rest of the retina.

Foveal acuity of vision is acknowledged in the art, as follows:

U.S. Pat. No. 4,513,317 (Retinally stabilized differential resolution television display, Ruoff Jr. and Carl F., 1985) describes a remote television viewing system employing an eye tracker, wherein a small region of the image appears in high resolution, and the remainder of the image appears in low resolution. The eye tracker monitors the position of the viewer's line of sight. The eye tracker position data is transmitted to the remote television camera and control. Both the remote camera and television display are adapted to have selectable high-resolution and low-resolution raster scan modes. The position data from the eye tracker is used to determine the point at which the high-resolution scan is to commence. The video data defining the observed image is encoded in a novel format, wherein in each data field, the data representing the position of the high-resolution region of predetermined size appears first, followed by the high-resolution zone video data and then the low-resolution region data. As the viewer's line of sight relative to the displayed image changes, the position of the high-resolution region changes to track the viewer's line of sight.

U.S. Pat. No. 5,422,653 (Passive virtual reality, Maguire Jr. and Francis J., 1995) discloses a method and apparatus for providing, in response to image signals originating in an object space, mixed image signals for providing non-uniform resolution images for stimulating simulated active percepts for passive perception by a viewer in an image space. The image& have a highly detailed component which his its image content changed according to changes in the direction of a simulated observer's eye position in an object space. The images may be provided stereoscopically. The images may be provided at various apparent distances, and the relationship between accommodation and convergence may be preserved. Audio waves for directionally simulating that which would be heard by the simulated observer may be provided.

WO 00/79,759 A1 (Transmission and display of video data, Ritter Rudolf and Lauper Eric, 2000) discloses an invention relates to a system and method for transmitting and displaying video data and to a communication terminal and an appropriate central video unit. Users can request and receive video data from the central video unit using communication terminals, in particular, mobile communication terminasl via a telecommunication network, in particular, a mobile telephone network. Image signals corresponding to the received video data are projected onto the retina of the user by a virtual retina display device of the communication terminal, whereby the current eye positions of the user are determined in the communication terminal and are transmitted to the central video unit. Said central video unit comprises a video filter module which filters the aforementioned video data before its transmission, based on the received current eye positions, in such a way that outer image areas corresponding to the video data, which are projected onto the retina outside the fovea have a lower resolution than the inter image areas corresponding to the video data, which are projected onto the fovea of the retina. Accordingly, the filtered video data contains a smaller amount of data than unfiltered video data.

Some physiological and cognitive laws are known that describe the behavior and perception of vision.

The Purkinje-Sanson effect describes the fact that three of the reflective surfaces of the eye (the cornea, the anterior face of the lens and the posterior face of the lens) act as half-mirrors, projecting three images onto the retina. Said three images are perceived by the brain as a single image.

Helmholtz' definition of field of vision states that by the combined action of the two eyes the field of vision is considerably enlarged. The combined visual fields form approximately a hemisphere, which is a wider field of operation than that of any artificial optical instrument.

The Ives-Cobb effect states that if two images are focused upon one single cone only one impulse is created which is coed by one optic nerve fiber to one definite area of the visual cortex (occipital lobe) of the brain. The activity of this one optical area gives rise to one, and only one, visual sensation. If the two images fall upon two neighboring cones the result is a fusion of the two impressions. But if the two images fall upon two cones separated by a third cone, than the observer sees two distinct points of light.

Binoculus (Cyclopean eye): In the case of distal diplopia, he two retinal images may be attributed to a single virtual eye located midway between the two eyes. The binoculus retina may by viewed as the merging of the two retinas whose two retinal images are cerebrally merged to a single image.

There is a need in the art to provide for an improved method and system for generating an image or succession of images on the fovea and retina of the viewer's eye(s), inter alia in order to reduce the transmission volume of data and consequently, the transmission bandwidth.

### SUMMARY OF THE INVENTION

The invention provides for a method for the generation of at least one image on a selectable part of the retina of a viewer's eye, to form an ocular image thereon, comprising:

(a) receiving an image being composed of a number of image elements that correspond to the number of cone photoreceptors of a viewer's fovea, so as to constitute a received image;

(b) displaying said received image or derivative thereof, so as to constitute a displayed image;

(c) projecting said displayed image or derivative thereof onto a viewer's fovea area, so as to constitute a foveal image, such that a number of image elements of said foveal image corresponds to the number of cone photoreceptors of the viewer's fovea; and

(d) projecting said displayed image or derivative thereof onto a viewer's retina so as to constitute a retinal image.

The invention further provides for a method for the generation of at least one image on a selectable part of the retina of a viewer's eye, to form an ocular image thereon, comprising:

(a) scaling down a source image into an image being composed of a number of image elements that correspond to the number of cone photoreceptors of a viewer's fovea and transmitting the image;

(b) receiving the image;

(c) displaying said received image or derivative thereof, so as to constitute a displayed image;

(d) projecting said displayed image or derivative thereof onto a viewer's fovea so as to constitute a foveal image, such that a number of image elements of said foveal image corresponds to the number of cone photoreceptors of the viewer's fovea; and

(e) projecting said displayed image or derivative thereof onto a viewer's retina so as to constitute a retinal image.

Still further, the invention provides for a method for transmission of high fidelity images over a low bandwidth communication channel, comprising performing the following in respect of each one of said images:

(a) scaling down the image into an image being composed of a number of image elements that correspond to the number of cone photoreceptors of a viewer's fovea and transmitting the image over the narrow band communication channel;

(b) receiving the image;

(c) generating the image or derivative thereof onto a viewer's fovea area so as to constitute a foveal image, such it a number of image elements of said foveal image corresponds to the number of cone photoreceptors of the viewer's fovea; and

(d) generating said displayed image or derivative thereof onto a viewer's retina so as to constitute a retinal image.

Yet further, the invention provides for a method for the generation of an image to form an ocular image on the fovea of an viewer's eye, the method comprising:

(a) displaying at image composed of a number of image elements that correspond to the number of cone photoreceptors of the viewer's fovea; and

(b) projecting said image onto the viewer's fovea.

The invention further provides for a method for the generation of at least one image on a selectable part of the retina of a viewer's eye, to form an ocular image thereon, comprising:

scaling down a source image into an image being composed of a number of image elements that correspond to the number of cone photoreceptors of a viewer's fovea and transmitting the image.

The invention further provides for a system for the generation of at least one image on a selectable part of the retina of a viewer's eye, to form an ocular image thereon, comprising:

receiver, receiving an image being composed of a number of image elements that correspond to the number of cone photoreceptors of a viewer's fovea, so as to constitute a received image;

display, displaying said received image or derivative thereof, so as to constitute a displayed image;

an assembly for projecting said displayed image or derivative thereof onto a viewer's fovea area, so as to constitute a foveal image, such that a number of image elements of said foveal image corresponds to the number of cone photoreceptors of the viewer's fovea;

the assembly further projecting said displayed image or derivative thereof onto a viewer's retina so as to constitute a retinal image.

Still further, the invention provides for a system for the generation of at least one image on a selectable part of the retina of a viewer's eye, to form an ocular image thereon, comprising:

a device for scaling down a source image into an image being composed of a number of image elements that correspond to the number of cone photoreceptors of a viewer's fovea and transmitting the image.

Yet further, the invention provides for an apparatus for the forming of an image on a selectable part of the retina of a viewer's eye to form an ocular image thereon, comprising:

a surface positioned approximately normal to an optical path, said optical path including a section incident to a selectable point of the retina,

means for generating a displayed image on said surface;

an optical element positioned along said optical path for directing light rays emanating from said displayed image to the viewers' eye, to form said ocular image.

a corporally mountable housing for the mounting of said surface and said optical element on said viewer.

wherein said image is changeably formed:

substantially on the fovea, and

on any part of the retina.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a cross-sectional diagram of the human eye;

FIG. 2 is a schematic depiction of a conventionally formed image on the retina;

FIG. 3 is a cross-sectional detailed diagram of a portion of a human retina;

FIG. 4 is a schematic depiction of the gradient of cone density in the retina;

FIG. 5A is a schematic representation showing the superposition of constant retinal image pixel density in a first resolution, on the gradient of cone density in the retina;

FIG. 5B is a schematic representation showing the superposition of constant retinal image pixel density, in a second—higher resolution, on the gradient of cone density in the retina;

FIG. 6 is a schematic representation of a perceived image according to an embodiment of the invention;

FIG. 7 is a schematic representation of a display screen for the alternating display of retinal and foveal images, in accordance with one embodiment of the invention;

FIG. 8 is a schematic representation of an ocularly focused projection system, constructed in accordance with one embodiment of the present invention;

FIG. 9 is a schematic representation of an ocularly focused projection system, constructed in accordance with another embodiment of the present invention;

FIG. 10 is a flow diagram showing pixel dilution before transmission, in accordance with an embodiment of the invention;

FIGS. 11A, B is a schematic representation of the ideal cone photoreceptors and of photons impinging on them.

FIG. 12 is a flow diagram showing frame re-sampling before transmission, in accordance with an embodiment of the invention;

FIG. 13 is a flow diagram showing regeneration of image frames, in accordance with an embodiment of the invention;

FIGS. 14A, B are schematic representations of projecting two images onto the viewer's eyes for recycling a stereoscopic perception.

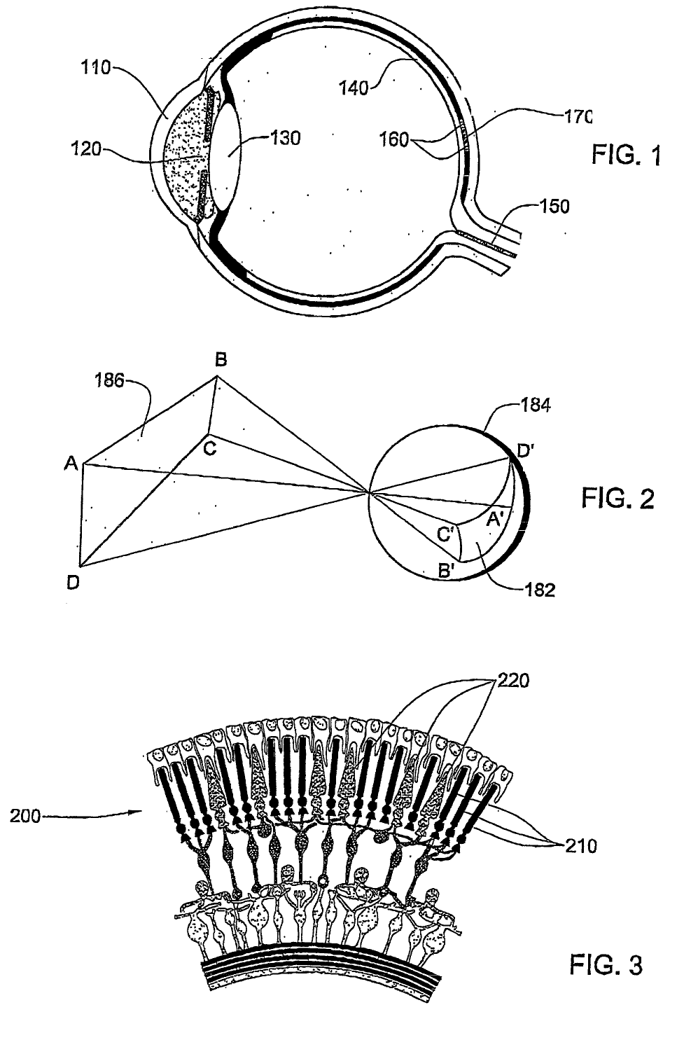

### DETAILED DESCRIPTION OF THE INVENTION

Note that the term fovea area should be construed as encompassing the fovea, a major portion of the fovea, or the fovea and certain neighboring macular area. For convenience, the description below refers to fovea, however, it is applicable also to other instances of the fovea area.

Whereas the description below refers to the specific usage of pixels, those versed in the art will readily appreciate that the invention is likewise applicable to any image elements. Note that the term pixel is only one example of an image element that may be utilized by the invention.

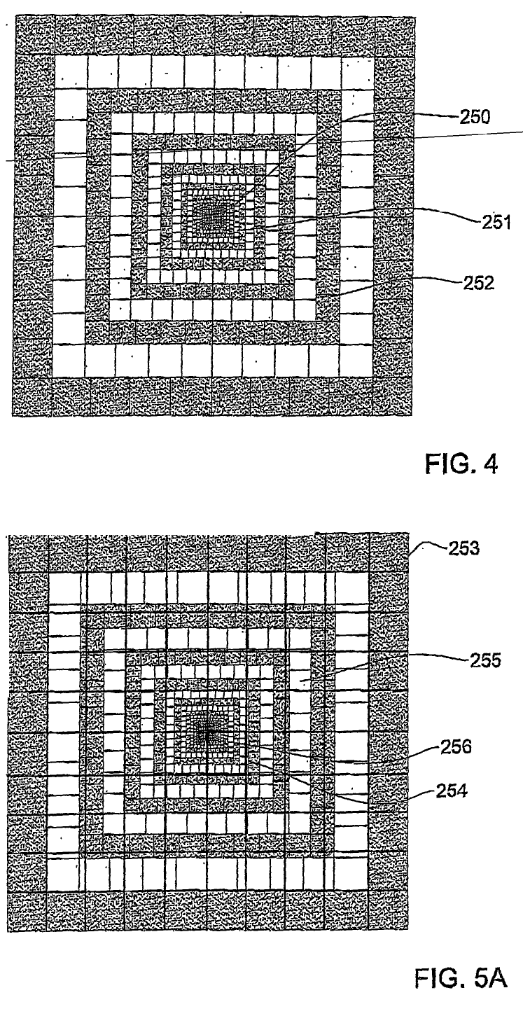

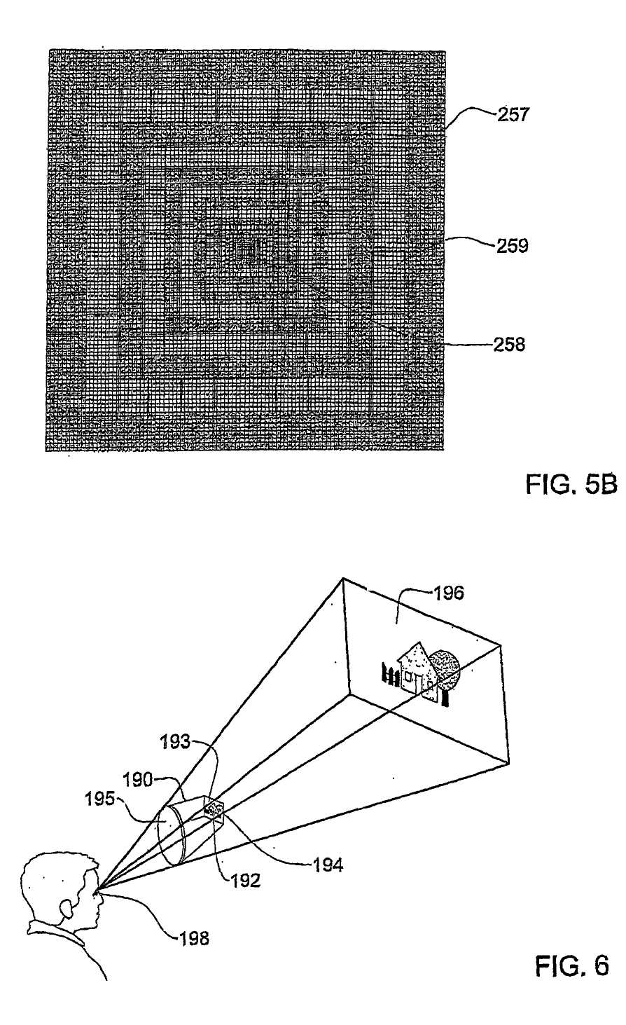

The present invention relates to a system that relies on a combination of the superior visual properties of the fovea and various properties of other parts of the retina, as well as visual perception properties of the brain in order to generate high quality perception of images transmittable over low bandwidth communication channels, e.g. conventional telephone lines and/or any other communication channel operating at a bandwidth of a least 20 Kbit per second. Turning at first to FIG. 5A, which schematically represents superposing a typical constant pixel density display device 253 (shown as large squares and bold edges), such as a TV screen, onto the widely variable retinal cone density that was shown in FIG. 4. Note that the cones' gradient is shown, as squares delimited by thin lines. The constant pixel density of the matrix 253 in FIG. 5A is comparably low; so as to approximately correspond with tie average cone density in most of the retinal area 255. According to FIG. 5A, a display technology utilizing low constant pixel density does not take advantage of the potentially higher quality and acuity of the fovea area. For example, pixel 254 in the constant pixel density display device 253 overlaps many foveal pixels and extends over approximately a quarter of the fovea 256.

FIG. 5B represents superposing a higher density constant pixel matrix 257 onto the widely variable retinal cone density represented by FIG. 4. The constant pixel density represented by matrix 257 is denser than the one represented in FIG. 5A by matrix 253, and is designed to relate the cone density in the fovea 258. FIG. 5B emphasizes that by using a higher pixel density, it is possible to take advantage of the foveal acuity of vision. However, in most of the retinal surface the cone density is drastically lower than the pixel density represented by matrix 257, thus many displayed pixels are not sensed or perceived, i.e. many displayed pixels are lost. For example, many pixels in the constant pixels density display device 257 impinge onto the single retinal cone area 259.

According to the Ives-Cobb effect, a cone creates only one output even if illuminated by more than one source simultaneously. Therefore the high retinal density is higher than the density required to effectively stimulate the retinal cones, thereby rendering some of the pixels redundant, and, consequently, requiring an undue bandwidth for transmission of the redundant pixels.

The Ives-Cobb effect also explains that there is a redundancy in using pixel density higher than the cones' density in the fovea.

As specified in the background of the invention, the stimulation of the fovea area is required to produce the best, most accurate and acute vision possible in normal daylight conditions. According to one embodiment of the invention, projecting a full image onto the fovea, referred to hereinbelow as foveal projection, allows the viewer to perceive a full and detailed image, his perception being characterized by the foveal high quality of vision.

By another embodiment, a second image, larger in size than the foveal image and corresponding to it, is also projected to the viewer's retina of one or both eyes, forming, thus, a retinal projection. According to the Purkinje-Sanson effect, the visual cortex:

Accepts the projected image from both eyes.

The foveal image with its high acuity and small size.

The retinal image with reduced acuity but larger in size

The two images are merged into a single perceived image in which:

The size is determined by that of the retinal image.

The acuity is defined by the foveal image.

Thus receiving a single, larger image where:

By modifying the retinal projection area it is possible to control the size of the perceived image.

While the quality of the perceived image is defined by that of the foveal image.

As the fovea covers a small area, subtending a small angular field of view of the eye, rapid eye movements and the Hehmholtz effects are required to accurately see objects subtending larger angles, therefore effectively covering a larger area than the fovea area. In those cases when the rapid eye movements occur, this allows the foveal image in yet another embodiment of the invention to occupy a slightly larger area then the area covered by the fovea.

Attention now is drawn to FIG. 6, which illustrates a schematic representation of a perceived image. A image 192, received from a communication channel (not shown) is displayed. As shown, light rays 190 emanating from the surface 193 of projector 194, further propagate through the displayed image 192 and arc projected onto the viewer's fovea. Light rays 190 are focused by a suitable arrangement of optics (schematically represented by lens 195), onto the viewer's eye(s) 198, thereby enabling the viewer to perceive a large apparent object 196 of the displayed image 192. By this embodiment, surface 193 is approximately perpendicular to an optical path extending from the fovea. Displayed image 192 can be generated by different means on surface 193. In one example, projector 194 has an LCD incorporating a large number of pixels. By another embodiment, projector 194 includes a screen, the surface 193 of which is reflective and on which an image is projected. Also, surface 193 may be modified to compensate for defects in the optical unit or the user's visual defects, such as corneal or lens deformations. The pixels or any other image element of projector 194 may controllably emit light, or they may controllably modify light reflected from them or transmitted through them by front or by back illumination to create a desired displayed image 192. Note that the pixel density in projector 194 generating displayed image 192 need not be constant. Any other means for selectably changing the spectrum of the light reaching projector 194, and emanated from it towards a viewer's eye (for example by some elements incorporated in it), could also be utilized to generate desired images on surface 193. Projector 194 may be constructed as part of a cranially supported device, similar to safety goggles and incorporating optics schematically represented by lens 195. Other elements such as electronic units could be located elsewhere, for example, as part of a body-mounted unit.

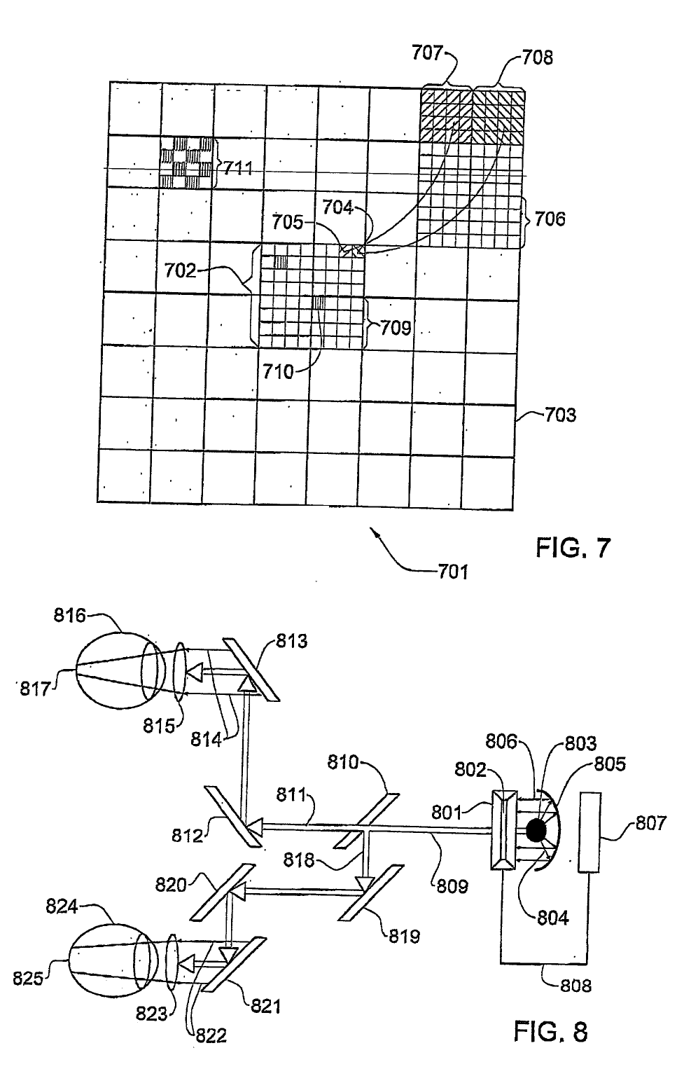

Turning now to FIG. 7, there is shown schematically, a display surface 701 for the alternating display of retinal and foveal images. A small image is reflected upon or transmitted from a small sub-surface 702 of surface 701. The small image is projected onto the fovea area, and therefore sub-surface 702 of surface 701 is designated as foveal display area 702 of surface 701.

A magnified image is reflected upon or transmitted from the surface 703. By one embodiment, surface 703 includes foveal display surface 702, and is therefore identical to surface 701. By another embodiment, surface 703 excludes foveal display surface 702, and includes the rest of surface 701. By yet another embodiment, surface 703 includes parts of foveal display surface 702.

The magnified image is projected onto the viewer's retina, therefore surface 703 is designated as retinal display surface. The displays on surfaces. 702 and 703 form two corresponding ocular images, preferably foveal image and retinal image, respectively, used to create the foveal and retinal projections, respectively. Both images are alternately reflected upon or transmitted from retinal image surface 703 and foveal display surface 702.

By one embodiment of the invention, the images reflected upon or transmitted from the retinal display surface 703 and the foveal display surface 702 are the same image but of different sizes. By another embodiment of the invention, the images are not the same, but rather one is a derivative of the other, say, one is obtained by applying a computational manipulation to the other.

By yet another embodiment the images reflected upon or transmitted from to the retinal display surface 703 and the foveal display surface 702 are of diffident images and different sizes.

Reverting to the embodiment of FIG. 7, the foveal display surface 702 is located in the center of display surface 701. Foveal display surface 702 occupies one quarter of the width of display surface 701 and one sixteenth of its area. The pixels in the foveal display 'surface 702, e.g. pixels 704 and 705, alternatively take part in both foveal display surface 702 and retinal display surface 703.

By another embodiment, display surface 701 includes a large number of contiguous, equal size pixels, e.g. pixels 704 and 705.

The pixels of retinal display surface 703 are divided into contiguous groups 706. Pixel groups 706 include an assembly of four by four pixels, simultaneously activated.

By other embodiments, several of pixel groups 706 may be of a different size or shape than the others, and the ratio between corresponding retinal and foveal pixel size and retinal and foveal pixel group size need not be constant. FIG. 7 illustrates only equal size groups 706.

In the embodiment illustrated by FIG. 7, each one of groups 706 correspond to one pixel in foveal display surface 702, i.e. each pixel in each group 706 has the same binary value as the corresponding pixel in foveal display surface 702. For example, pixel 705 in foveal display surface 702 corresponds to pixel group 707 in retinal display surface 703, whereas pixel 704 in foveal display surface 702 corresponds to pixel group 708 in retinal display surface 703. A possible exception exists concerning those groups of retinal display surface 703 that are also included in foveal display surface 702, such as group 709 that corresponds to inner pixel 710. Groups 706 may also include groups such as pixel group 711, wherein only one half of the pixels that are included in it are activated. Other embodiments are possible in which other arrangement of partly activated pixel groups 706 are described.

FIG. 8 is a generalized schematic representation of an ocularly focused projection system, constructed in accordance with one embodiment of the present invention. By this embodiment, the foveal and/or retinal images are formed from a displayed image generated on surface 801 of body 802.

In operation, light source 803 radiates light rays 804 in a spherical manner, i.e. in all directions. Rays 804 that are directed to the back side of the light source propagate towards a concave mirror 805, reflected back towards the body 802 in the form of, basically, parallel light rays 806.

By one embodiment, body 802 receives images transmitted to it by a remote side, for display by means of image elements. In another embodiment, electronic unit 807 receives the images transmitted to it by a remote side. The image elements are controlled by electronic unit 807 via conductor 808 to controllably modify the color of the transmitted light rays 806, generating the images.

The tracing of one ray 809 among rays 806, transmitted through the image elements of body 802, is shown. Ray 809 is split by a beam splitter 810 into split rays 811 and 818. The split rays 811 and 818 propagate by their respective optical elements, represented in this embodiment by mirrors 812, 813 and lens 815 towards eye 816, and by mirrors 819, 820, 821 and lens 823 towards the other eye 824. To firer emphasize the basically parallel nature of light rays 811 and 818, other light rays 814 and 822 respectively are also drawn in parallel to them. The parallel light rays 811 and 818 flier propagate to meet lenses 815 and 823 respectively, where they are focused onto the appropriate area of the eye's retina, i.e. the fovea area or a wider area of the retina, to form the appropriate image, i.e. foveal image or retinal image respectively. The character of lenses 815 and 823, i.e. the lenses' focus intensity, determines the character of the image formed on eyes 816 and 824 respectively, i.e. foveal or retinal image. To change the formed image character from foveal image to retinal image or vice versa, a different lens with the corresponding focus intensity must replace lens 815 or 823. By this embodiment, light rays 811 and 818 are to be focused by lenses 815 and 823 respectively onto the respective parts of the fovea of eye 816, to form a foveal image 817, and of the retina of eye 824, to, form a retinal image 825.

Similar optical paths exist for other rays transmitted through other image elements of body 802 and are focused onto different parts of the fovea and the retina, to form images (not shown) on the fovea and retina, of the displayed images generated on surface 801. The foveal images lead to the highest perceived acuity of the displayed images from which they are derived while the retinal images increase the field of view. Both images are merged into one image by their cerebral processing, according to the Purkinje-Sanson effect, thereby creating the combined virtual image.

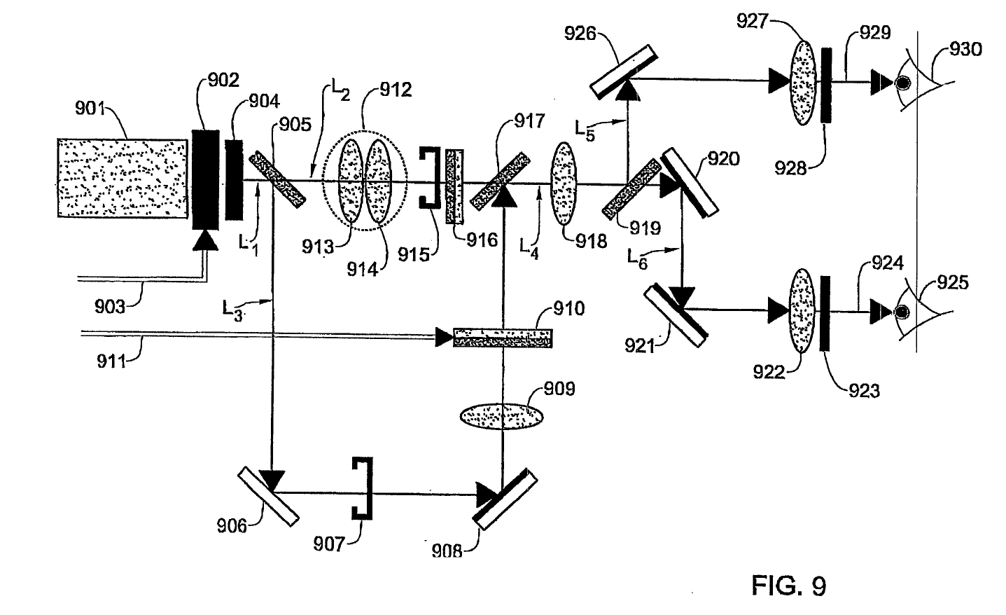

FIG. 9 describes an embodiment of the invention. As may be recalled, in the embodiment of FIG. 7 foveal and retinal projections originate from respective foveal and retinal images. In contrast, in the embodiment of FIG. 9 the foveal and retinal projections are formed by means of optical assembly, from a single image displayed on a microdisplay 902. The optical assembly is electronically controlled to alternate between foveal and retinal projections for each eye.

At the onset, images are received as an input 903 to the display system. The images may be transmitted from a remote side, where they are produced, say as a succession of images, for example, video frames (by, e.g. a DVD player). Reverting to the receiving side, light rays that emanate, from a light source 901 located at the back of microdisplay 902 are transmitted through the microdisplay 902 reaching polarizer 904, the objective of which being to assure that the image light is of a specific linear polarization (ordinary polarization component TE, or extraordinary polarization component TM). Note that the displayed image is not necessarily identical to the received one, i.e. it may be a derivative thereof (say, one is obtained by applying a computational manipulation to the other).

Note that the terms video images or video frames are only one example of a succession of images that may be utilized by the invention

The polarizer 904 is has to be compatible with the polarization of the light emitted by light source 901, i.e. to allow passage therethrough of predetermined linear polarization, for example TE or TM polarization components. To correct the small deviation of the image light coming from the microdisplay 902, the polarizer 904 is positioned close to the microdisplay 902. The polarizer 904 creates the initial light component L₁.

The initial light component L₁ impinges on beam splitter 905, creating two light components L₂ and L₃, independently performing optical manipulations for creating the retinal image (hereinbelow retinal light component L₂) and the foveal image (hereinbelow foveal light component L₃).

In order to appropriately re-combine the two light components L₂ and L₃, two front surface mirrors 906 and 908 are included in the propagation path of light component L₃, deflecting the light paths by 90° each.

A light-expanding unit 912 (typically formed by two lenses 913 and 914 appropriately designed and oriented with respect to each other) determines the size of the retinal image created by light component L₂.

The light intensities of two light components L₂ and L₃ propagating towards, respectively, numeric apertures 915 and 907 are appropriately adjusted by variable attenuators inserted into the numeric apertures. Light component L₃ propagates her towards a lens 909, which determines the size of the foveal image created by light component L₃.

The light components L₂ and L₃ further propagate through e.g. Ferro-electric Liquid Crystal assembly (FLC assembly including an FLC component and a polarizer, known per-se) 916 and 910 respectively, which may be electrically controlled and aim at controlling the polarization and/or acting as a shutter. FLC assembly 916 does not receive an electrical command and therefore operates as a passive element for rotating the polarization of light component L₂ by 90°. FLC assembly 910 further receives appropriate electrical command 911, therefore operating as a shutter, which can block or pass the image carried by light component L₃. Unlike FLC assembly 916, FLC assembly 910 does not rotate the image any further.

The light components L₂ and L₃ are re-combined to a fourth light component L₄ by a second beam splitter 917. The combined light component L₄ contains both foveal and retinal images, being polarized to two different polarizations, e.g. when the retinal image is characterized by TM polarization the foveal image is characterized by TE polarization.

Light component L₄ further propagates through a lens 918 used to adjust the image to the desired size. Light component L₄ further impinges on a third beam splitter 919 splitting the light component L₄ to two separate light components L₅ and L₆ which propagate separately to the viewer's eyes 930 and 925, respectively. To direct light components L₅ and L₆ to the eyes, front surface mirrors 920, 921 (for eye 925) and, 926 (for eye 930) are used in order to deflect the light path by 90° each. In front of each eye 925 and 930, there are disposed lenses 922 and 927 respectively, reducing the image size to correspond to the required projection size. Polarizers 923 and 928 allow only TM or TE light rays to propagate through them. Depending on whether they allow TM or TE light propagation, foveal/retinal projections are allowed to reach the eyes.

For a better understanding of the foregoing, consider the following example. Polarizer 904 propagates TE polarized light rays (L₁). FLC assembly 916 rotates the retinal light component L₂ by 90° rendering it TM polarized. FLC assembly 910 enabling TE polarized foveal light component L₃ to pass (without affecting its polarization). Accordingly, the re-combined light component L₄ includes a TE polarized foveal component and a TM polarized retinal component. L₄ is split to L₅ and L₆, each including the same components. Polarizer 928 propagates TE polarized light rays, therefore eye 930 receives the TE polarized light components, i.e., the foveal projection. Polarizer 923 propagates TM polarized light rays, therefore eye 925 receives the TM polarized light components, i.e. the retinal projection.

To alternate the foveal and retinal projections (i.e. for eye 930 to receive retinal projection and for eye 925 to receive foveal projection), the polarizations of polarizers 928 and 923 are suitably rotated.

Note, the TE polarized foveal projection from the last example described above is performed on the viewer's fovea.

It should be noted that the embodiments described with reference to FIGS. 8 and 9 realize two out of many possible variants of realizing projection of displayed images on the fovea area and the retina. The invention is by no means bound by these specific implementations.

Having described how to project an image onto the fovea area (foveal projection 924) and on the retina (retinal projection 929), there follows now few non-limiting embodiments for selectively projecting images on the fovea area and the retina of one or two of the eyes, controlled by polarizers 904, 923 and 928 and by FLC assemblies 910 and 916. Note that controlling by means of polarizers is only one non-limiting example.

Thus by one embodiment, said foveal image and said retinal image being projected to the same viewer's eye, e.g. 925.

By another embodiment, said foveal image and said retinal image being projected simultaneously (to the same eye or to different eyes).

By still another embodiment, said foveal image being projected before said retinal image (to the same eye or to different eyes).

By still another embodiment, said foveal image being projected after said retinal image (to the same eye or to different eyes).

Note that, for simplicity, the description with reference to FIG. 9, concerned mainly a projection of a single image (to yield a retinal and a foveal projections).

Those versed in the art will readily appreciate that the embodiment described with reference to FIGS. 8 and 9, is likewise applicable to a succession of images e.g. a series of video frames.

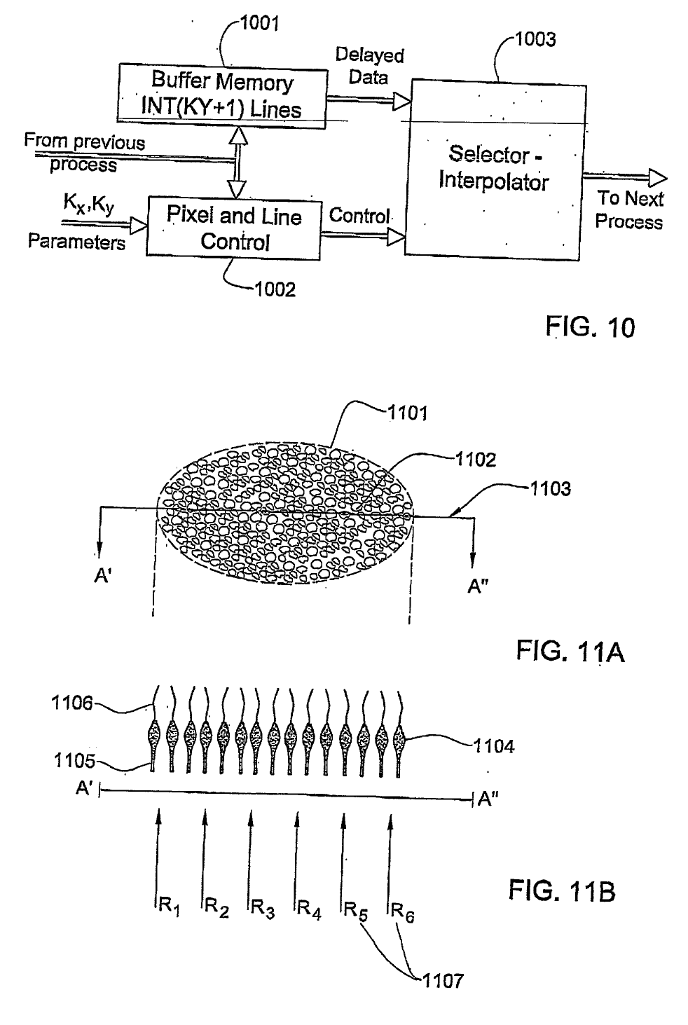

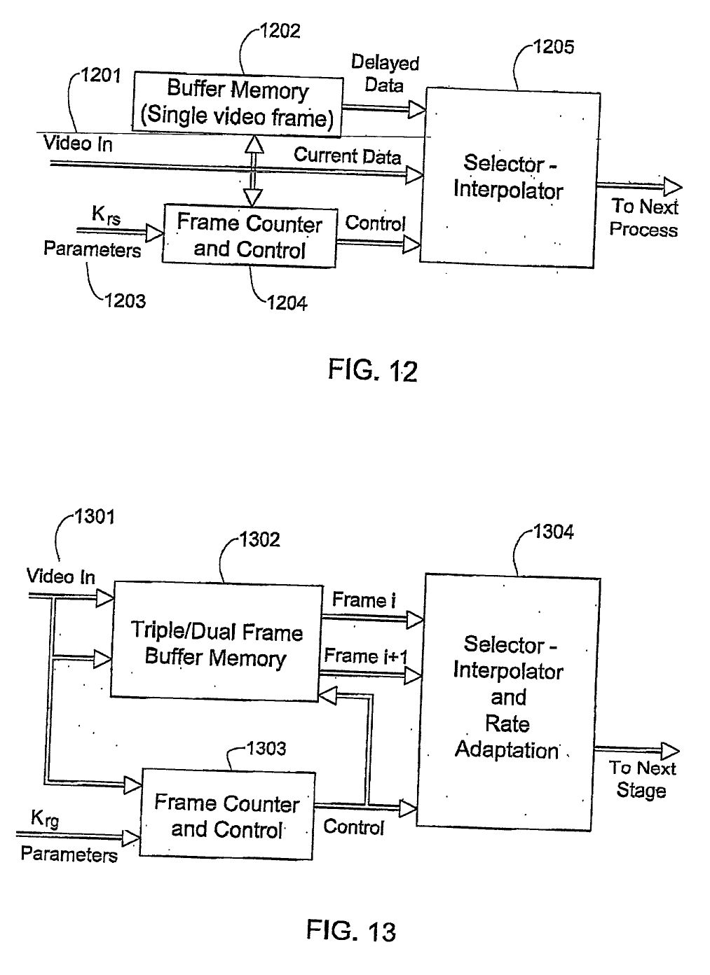

Having described the receiving side there follows a description of the transmitting side with reference to FIG. 10 and FIG. 12.

Note that the terms ‘scale down’ or ‘scaling down’ of an image or of a succession of images refers to scaling down in terms of the number of pixels, therefore reducing the bandwidth required for the image or images' transmission.

Bearing this in mind, at the transmitting side, source images are scaled down to have a number of pixels that corresponds (e.g. being a predefined value relating to the typical number of cone photoreceptors) to the number of cone photoreceptors in the fovea. The scaled down image or succession of images is transmitted via communication channels, such as telephone lines characterized by a transmission capacity of, say, 56 Kbit per second or cellular communication lines that have a transmission capacity of 20 Kbit per second, preferably by using standard communication equipment. This allows the use of low bandwidth communication channels (i.e. communication channels supporting no less than 20 Kbit per second), such as modems and other currently available equipment for the generation of quality video displayed images compatible with the high quality foveal resolution, by combining current image compression techniques with a reduced rate displayed image.

By one embodiment, the scaling down of an image requires the dilution of the number of pixels in the original image, from e.g. 307,000 pixels (VGA), to e.g. about 30,000 pixels, corresponding to the number of cone photoreceptors in the fovea. The term “corresponding” should be construed as not necessarily one to one correspondence between the number of pixels and the number of cone photoreceptors in the fovea. For example, if the known H.320 standard is use the number of pixels (that correspond to the number of cone photoreceptors) per frame is 26,000.

In yet another embodiment, scaling down of an image can be performed by frame re-sampling, in a way that reduces the rate by which images, composing a succession of images are transmitted.

Note that the term ‘frame’ designates one image within a succession of images.

Pixel dilution can be performed by different methods. For example, if the source (non-diluted) image's pixels are spread over an XY matrix, it is possible to define two constants Kx and Ky. Pixels on the matrix should be grouped by the. Kx and Ky factors, such that each group should be composed of Kx * Ky pixels. In the diluted image, each pixel represents a group of pixels in the source image such that all the groups (and therefore all the pixels) in the source image are represented. Each pixel in the diluted image is created, for example as a weighted average of all pixels in the group, or, by way of another example, as a selection from the pixels in the group, according to some predefined criteria. In the case of Kx, Ky which are non-integer numbers, interpolation must be used. For example: let Kx=Ky=2.5. That means that one pixel must be computed based on a matrix of 2.5*2.5 pixels or more intuitively: four pixels will be computed from a group of 5*5 pixels, which is actually composed of four groups of 2.5*2.5 pixels. The following computation demonstrates computing these four pixels:

ComputedPixels(1,1)=SourcePixel(1,1)

ComputedPixels(1,2)=(SourcePixel(1,3)+SourcePixel(1, 4))/2

ComputedPixels(2,1)=(SourcePixel(3,1)+SourcePixel(4, 1))/2

ComputedPixels(2,2)=(SourcePixel(3,3)+SourcePixel(3,4)+SourcePixel(4,3)+SourcePixel(4,4))/4

As shown here, ComputedPixel(1,2), for example, is an average, or interpolation, of the two pixels SourcePixel(1,3) and SourcePixel(1,4)).

The example described above is by no means binding.

Attention is now drawn to FIGS. 10 illustrating a flow diagram showing pixel dilution before transmission, according to an embodiment of the invention. The pixels composing INT(Ky+1) lines are fed to a buffer memory 1001. The Kx, Ky parameters are fed to the Pixel and Line control 1002. In the case of integer Kx, Ky—a selector 1003 is used to chose the above referred to SourcePixels that will serve as the ComputedPixel in the scaled down image. Otherwise, in the case of non-integer Kx, Ky an Interpolator 1003 will be used instead, to perform averaging of pixels as required.

As specified above, there is not necessarily a one-to-one correspondence between the number of pixels and the number of cone photoreceptors in the fovea. In this context, there follows an example (with reference to FIGS. 11A, B) of a one to four ratio between the number of pixels and the number of cone photoreceptors in the fovea. Note that despite this ratio, the acuity of vision in the fovea is maintained.

Thus, FIG. 11A illustrates a schematic representation of the foveal cone photoreceptors and of photons impinging on them. The, figure depicts a plan view of a small part of the fovea 1101. The cones are tightly packed 1102 and form a very dense mosaic.

FIG. 11B shows a cross-sectional view along A′-A″ line 1103 of FIG. 11A, where a group of cones are shown. 1104 is a typical cell body of a cone photoreceptor cell. Member 1105 of the photoreceptor cell functions as a photo sensor, while member 1106 connects the done photoreceptor cell to the nervous system. Note that a typical average foveal cone diameter is 0.002 mm.

Assuming, by this specific example, that the size of a pixel is substantially the same as that of the cone photoreceptor, and further bearing in mind that there is a low probability that a pixel would fully coincide with a single cone, it readily arises that, as a rule, a pixel would impinge on two neighboring cones. Bearing this in mind, and further assuming that the pixel density is the same as that of the cones, there is high probability that two neighboring pixels would impinge on a single cone. Recalling also the Ives-Cobb effect, the latter two impinging pixels would generate only one output impulse from the cone they impinge on, giving rise to undue redundancy. Accordingly, the pixel density can be diluted. In other words, it is possible to reduce both the number of lines in a frame by a factor of two, and the number of pixels in a line by a factor of two, thus reducing the number of the communicated and displayed pixels by a factor of four.

The invention is, of course, not bound by this specific dilution technique. Further reduction in the transmitted data rate may be achieved by forming a displayed image that uses different factors for the pixel dilution, such as each third cone, resulting in dilution by factor of nine. By a specific example, the dilution factor is determined such that the number of pixels will meet the stipulations of the H.320 standard.

In another embodiment of the invention, a succession of images are subject to scaling down by other techniques, e.g. by re-sampling of video frames. It is also known that while viewing a succession of images, if the frequency of tie perceived image frames at the fovea is reduced to a lower frequency than 10 Hz-13 Hz, the viewer notices, in case of faster motion, jumps between successive frames, i.e. discrete changes between successive frames. Currently, the generation of movies and TV images uses frequencies such as 25 Hz, 30 Hz or even 100 Hz and possibly higher. Therefore, reducing the transmission frequency to, say, 13 Hz would result in the smooth motion of the succession of images in the viewer's eyes, while reducing the required bandwidth by a factor of about 2.5 (as compared to the conventional 30 frames per second) or even by about 8 (such as in the case of comparing against a 100 Hz transmission).

The frames' re-sampling can be performed by different methods. For example, let Krs be the re-sampling factor, i.e. according to which re-sampling is performed. One frame is to be generated from every Krs source frame. In the case of an integer Krs, one frame (e.g. the first) of every consecutive Krs frames is used. In the case of a non-integer Krs, for example Krs=3.5, interpolation must be used. In the said example of Krs=3.5, one frame should be transmitted for every 3.5 source frames, or more intuitively: two frames should be transmitted for every 7 source frames. The following computation demonstrates computing these two frames:

ComputedFrame(1)=SourceFrame(1)

ComputedFrame(2)=(SourceFrame(4)+SourceFrame(5))/2

As shown in the last example, when Krs=3.5, the ComputedFrame(2) is the average or interpolation of SourceFrame(4) and SourceFrame(5). The averaging of frames can be performed, by a non-limiting example, on a pixel-by-pixel basis.

FIG. 12 illustrates a flow diagram showing frame re-sampling before transmission, in accordance with an embodiment of the invention. Source video frames 1201 are fed as input into a buffer memory 1202, storing one single fame at a time. The re-sampling factor Krs 1203 is fed into the Frame Counter and Control 1204. In the case of an integer Krs, selector 1205 is used to choose the SourceFrame that will serve as the ComputedFrames in the scaled down image. Otherwise, in the case of a non-integer Krs, an interpolator 1205 will be used instead, to perform averaging of frames as required.

Having described few non-limiting embodiments of scaling down procedures performed at the transmitting side, attention is drawn again to the receiving side. The receiving side would receive, display and project the image or succession of images in a manner that was described in detail with reference to FIGS. 7 to 9 above.

Note that on the receiving side, if the rate of the incoming image frames is so low that flickering may occur, it is possible to cope with the problem by, e.g. re-generating frames (at the receiving side) in a way that increases the frequency to a desired rate, e.g. 50 frames per second.

The frames' re-generation can be performed by different methods. For example, let Krg be the re-generation factor, i.e., according to which frames are re-generated to prevent flickering. Krg frames are generated from each frame received at the receiving side. In the case of an integer Krg, according to one embodiment of the invention, each received frame is replicated Krg times. According to another embodiment, interpolation may be used. For example, let Krg=3. In this case:

RegeneratedFrame(1)=ReceivedFrame(1)

RegeneratedFrame(2)=(⅔) * ReceivedFrame(1)+(⅓) * ReceiveFrame(2)

RegeneratedFrame(3)=(⅓) * ReceivedFrame(1)+(⅔) * ReceivedFrame(2)

The above computation demonstrates that the first frame, i.e. RegeneratedFrame(1), out of the three resulted frames is identical to the received frame. The second resulted frame, i.e. RegeneratedFrame(2), resembles mainly the first received frame, and a little the second received frame. Finally the third resulted frame i.e. RegeneratedFrame(3), resembles mainly the second received frame, and slightly the first received frame. Therefore, the three resulting frames gradually shift from the first received frame to the second one. If Krg is non-integer, it is still possible, by one embodiment, to replicate each received frame by the truncation of Krg or (Krg+1), so as to generate integer numbers out of tie non-integer Krg. By yet another embodiment, it is possible to use interpolation to compute the Krg frames according to each received frame. The sample computation below demonstrates the interpolation computation for Krg=2.5, i.e. generating 2.5 out of each received frame, or more intuitively: generating 5 frames out of every two received frames:

RegeneratedFrame(1)=ReceivedFrame(1)

RegeneratedFrame(2)=(⅗) * ReceivedFrame(1)+(⅖) * ReceivedFrame(2)

RegeneratedFrame(3)=(⅕) * ReceivedFrame(1)+(⅘) * ReceivedFrame(2)

RegeneratedFrame(4)=(⅘) * ReceivedFrame(2)+(⅕) * ReceivedFrame(3)

RegeneratedFrame(5)=(⅖) * ReceivedFrame(2)+(⅗) * ReceivedFrame(3)

RegeneratedFrame(6)=ReceivedFrame(3)

The re-generated frames gradually resemble the second and later the third received image, when the sixth frame (which is the first of the next group of five images) is identical to the third received image.

FIG. 13 illustrates a flow diagram showing the regeneration of image frames, in accordance with an embodiment of the invention. Received video frames 1301 are fed as input to a buffer memory 1302, storing two or three frames as required. The re-generation factor Krg is fed into the Frame Counter and Control 1303. When the re-generated frame needs to be identical to any received frame, the selector 1304 is used. Whenever the re-generated frame should be an interpolation result, the interpolator 1303 is used instead. The re-generated rate is adapted to the required rate by the Rate Adaptation device 1303.

Having described the operation of the transmitting and receiving sides in accordance with several non-limiting embodiments of the invention, there follows a numeric example which illustrates the reduction in the required bandwidth. Thus, for TV frames transmitted in YIQ format, at 10 Hz, a pixel dilution factor of four in the number of pixels, and a 100 compression factor (e.g. MPEG-1), the nominal TV bandwidth of about 3.6 Mpixels per second is reduced to 3 Kpixels per second.

The calculation is:

3 Kpixel/sec=NominalTVBandwidth * FrameRateRation * PixelDilutionFactor * NormalCompressionRation=3,456 Kpixels/sec * (10/30) * (¼) * (1/100)

Where

NominalTVBandwidth=3,456 Kpixels/sec=480 pixels/line * 240 lines/frame * 30 frames/sec

In the case of RGB format (i.e. 3 Kbytes per pixel) the required bandwidth becomes 9 Kbyte/sec.

In TV transmission, 10-12 bit/pixel formats are used instead of RGB. Thus the required bandwidth does not pass 4.5 Kbyte/sec, i.e. 36 Kbit/sec.

These numbers are below the full capacity of regular telephone lines. In contrast in accordance with conventional TV transmission techniques, the required bandwidth would be 5 Mbit/sec. There are applications, which require considerably higher bandwidth.

The invention is of course not bound by this specific example. It has therefore been shown that an ocular display system according to this invention can produce a TV quality image while using standard telephone lines for their communication. This permits the use, by one embodiment, of inexpensive Internet technology to communicate data for high quality video display.

Other embodiments of the present invention support also image projection enabling stereoscopic perception by the viewer. In one embodiment, two cameras take photographs of the imaged object (or space), from two slightly different angles of sight, simulating two-eye vision when one eye watches the object from an angle slightly distal to the second eye. The two cameras are connected to the transmitting apparatus, in such a way that both transmit their images to it, alternatingly. The receiving apparatus is synchronized with the alternating cameras, in a way that only the right eye receives the projection when the right camera is transmitting its images, and alternatively, only the left eye receives the projection when the left camera transmits its images.

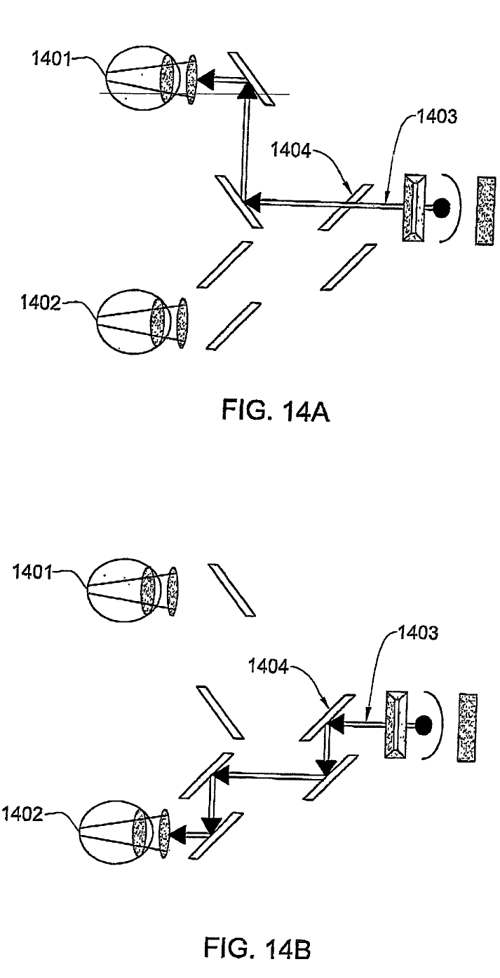

FIGS. 4A, B illustrate the projection of images on the viewer's right eye 1401 and left eye 1402 to form stereoscopic perception, according to the embodiment illustrated in FIG. 8. The light rays 1403, projecting the image, impinge a half-transparent mirror 1404. When the projected image is the right camera's image (FIG. 14A), the half transparent mirror 1404 turns to be fully transparent, and therefore the light rays 1403 do not deflect towards the left eye 1402. When the projected image is the left camera's image (FIG. 14B), the half-transparent mirror 1404 turns to be a mirror, preventing light rays 1403 from propagating forward towards the right eye 1401. The invention is, of course, not bound by the specific implementation described with reference to FIGS. 14A, B.

In the embodiment of FIGS. 14A, B the required bandwidth is multiplied by 2, as each eye must receive now transmission characterized by a frequency of, for example 50 frame's per second, therefore the total rate, for both eyes, reaches 100 frames per second.

In accordance with another embodiment of the invention, this problem is coped with by receiving a succession of images from only one camera, and computing the distal image, instead of receiving it from a second camera. The computation can be performed by methods and algorithms shown per se, see e.g. U.S. Pat. No. 5,821,943 (Apparatus and method for recreating and manipulating a 3D object based on a 2D projection thereof, Shashua Amnon, 1998), whose contents is incorporated herein by reference. By performing the said computation at the receiving side, it is possible to lower the required bandwidth back to the rates required while transmitting a non-stereoscopic succession of images.

Note that whereas the present invention concerns transmission of images, is if desired, other data such as video an/or text may also be transmitted. In the method claims that follow, alphabetic characters and Roman numerals used to designate claim steps are provided for convenience only and do not imply any particular order, of performing the steps.

The present invention has been described with a certain degree of particularity, and accordingly those versed in the art will readily appreciate that various alterations and modifications may be carried out without departing from the scope of the following claims:

1. A method for the generation of at least one image on a selectable part of the retina of a viewer's eye, to form an ocular image thereon, comprising:
(a) receiving an image being composed of a number of image elements that correspond to the number of cone photoreceptors of a viewer's fovea, so as to constitute a received image;
(b) displaying said received image or derivative thereof, so as to constitute a displayed image;
(c) projecting said displayed image or derivative thereof onto a viewer's fovea area, so as to constitute a foveal image, such that a number of image elements of said foveal image corresponds to the number of cone photoreceptors of the viewer's fovea; and
(d) projecting said displayed image or derivative thereof onto a viewer's retina so as to constitute a retinal image.

2. The method according to said claim 1, wherein said foveal and retinal images both include the same number of image elements.

3. The method according to said claim 1, wherein said foveal image includes larger number of image elements than the number of image elements included in said retinal image.

4. The method according to claim 1, wherein said foveal image being the image received in (a).

5. The method according to claim 1, wherein said retinal image being the image received in (a).

6. The method according to claim 1, wherein said number of image elements of said received image being substantially one fourth of said number of cone photoreceptors of the viewer's fovea.

7. The method according to claim 1, wherein said number of image elements of said foveal image being substantially one fourth of said number of cone photoreceptors of the viewer's fovea.

8. The method according to claim 1, wherein said number of image elements of said received image being the same as the number of cone photoreceptors of the viewer's fovea.

9. The method according to claim 1, wherein said number of image elements of said foveal image being the same as the number of cone photoreceptors of the viewer's fovea.

10. The method according to claim 1, wherein said image element being a pixel.

11. The method according to claim 1, wherein said foveal image and said retinal image being projected to the same viewer's eye.

12. The method according to claim 11, wherein said foveal image and said retinal image being projected simultaneously.

13. The method according to claim 11, wherein said foveal image being projected before said retinal image.

14. The method according to claim 11, wherein said foveal image being protected after said retinal image.

15. The method according to claim 1, wherein said foveal image and said retinal image being projected to different viewer's eyes, respectively.

16. The method according to claim 15, wherein said foveal image and said retinal image being projected simultaneously.

17. The method according to claim 15, wherein said foveal image being projected before said retinal image.

18. The method according to claim 15, wherein said foveal image being projected after said retinal image.

19. The method according to claim 1, wherein said foveal image and said retinal image being projected also to the other viewer's eye.

20. The method according to claim 1, wherein the image received in (a) is a view of an object from a given direction, and further comprising:
(e) computing an image to be a view of the same object as from a different direction and applying said (b) to (d) in respect of the computed image, so as to constitute a stereoscopic perception of said object by the other eye of the viewer.

21. The method according to claim 1, wherein the image received in (a) is a view of an object from a given direction, and further comprising:
repeating said (a) to (d) in respect of a received image of the same object from a different direction, so as to constitute a stereoscopic perception of said object by the other eye of the viewer.

22. The method according to claim 1, wherein said (a) to (d) are repeated in respect of each image in a succession of images.

23. A method according to claim 22 wherein said succession of received images being video images.

24. The method according to claim 22, wherein said images are received at a rate of at least 20 Hz.

25. The method according to claim 22, further comprising:
(f) regenerating at least one image so as to constitute, together with at least one received image, a succession of images; sad
(g) repeating said (b) to (d) in respect to each image of said succession of images.

26. The method according to claim 22, wherein at least two consecutive images from said succession of received images are views of the same object taken form different directions, so as to constitute a stereoscopic perception of said object by the viewer.

27. A method for the generation of at least one image on a selectable part of the retina of a viewer's eye, to form an ocular image thereon, comprising:
(a) scaling down a source image into an image being composed of a number of image elements that correspond to the number of cone photoreceptors of a viewer's fovea and transmitting the image;
(b) receiving the image;
(c) displaying said received image or derivative thereof, so as to constitute a displayed image;
(d) projecting said displayed image or derivative thereof onto a viewer's fovea so as to constitute a foveal image, such that a number of image elements of said foveal image corresponds to the number of cone photoreceptors of the viewer's fovea; and
(e) projecting said displayed image or derivative thereof onto a viewer's retina so as to constitute a retinal image.

28. A method according to claim 27 wherein said (a) further includes transmission of data other that said image.

29. A method for transmission of high fidelity images over a low bandwidth communication channel, comprising performing the following in respect of each one of said images:
(a) scaling down the image into an image being composed of a number of image elements that correspond to the number of cone photoreceptors of a viewer's fovea and transmitting the image over the narrow band communication channel;
(b) receiving the image;
(c) generating the image or derivative thereof onto a viewer's fovea area so as to constitute a foveal image, such that a number of image elements of said foveal image corresponds to the number of cone photoreceptors of the viewer's fovea; and
(d) generating said displayed image or derivative thereof onto a viewer's retina so as to constitute a retinal image.

30. A method according to claim 29 wherein said (a) further includes transmission of data other that said image.

31. The method according to claim 29, wherein said low bandwidth communication channel being a telephone line

32. The method according to claim 29, wherein said communication channel has a transmission capacity of at least 20 Kbit per second.

33. The method according to claim 32, wherein said communication channel being a telephone line and has a transmission capacity of 56 Kbit per second.

34. The method according to claim 32, wherein said communication channel being a cellular communication line and has a transmission capacity of 20 Kbit per second.

35. A method for the generation of an image to form an ocular image on the fovea of an viewer's eye, the method comprising:
(a) displaying an image composed of a number of image elements that correspond to the number of cone photoreceptors of the viewer's fovea; and
(b) projecting said image onto the viewer's fovea.

36. A method for the generation of at least one image on a selectable part of the retina of a viewer's eye, to form an ocular image thereon, comprising:
scaling down a source image into an image being composed of a number of image elements that correspond to the number of cone photoreceptors of a viewer's fovea and transmitting the image.

37. A method according to claim 36 further comprising transmitting data other than said image.

38. A system for the generation of at least one image on a selectable pat of the retina of a viewer's eye, to form an ocular image thereon, comprising:
receiver, receiving an image being composed of a number of image elements that correspond to the number of cone photoreceptors of a viewer's fovea, so as to constitute a received image;
display, displaying said received image or derivative thereof, so as to constitute a displayed image;
an assembly for projecting said displayed image or derivative thereof onto a viewer's fovea area, so as to constitute a foveal image, such that a number of image elements of said foveal image corresponds to the number of cone photoreceptors of the viewer's fovea;
the assembly further projecting said displayed image or derivative thereof onto a viewer's retina so as to constitute a retinal image.

39. A system for the generation of at least one image on a selectable part of the retina of a viewer's eye, to form an ocular image thereon, comprising:
a device for scaling down a source image into an image being composed of a number of image elements that correspond to the number of cone photoreceptors of a viewer's fovea and transmitting the image.

40. An apparatus for the forming of an image on a selectable part of the retina of a viewer's eye to form an ocular image thereon, comprising:
a surface positioned approximately normal to an optical path, said optical path including a section incident to a selectable point of the retina,
means for generating a displayed image an said surface;
an optical element positioned along said optical path for directing light rays emanating from said displayed image to the viewers' eye; to form said ocular image,
a corporally mountable housing for the mounting of said surface and said optical element on said viewer,
wherein said image is changeably formed:
substantially on the fovea, and
on any part of the retina.

41. The apparatus according to claim 40, wherein said surface for generating said displayed image thereon:
is part of a body comprising a plurality of image elements,
is in optical communication with said image elements.

42. The apparatus according to claim 41, wherein said surface for generating said displayed image is in functional communication with a source of video images to generate said displayed image thereon.

43. The apparatus according to claim 42, wherein said source of succession of images transmits data to generate at least 10 displayed images per second at a constant frequency.

44. The apparatus according to claim 40, wherein said surface for generating said displayed image thereon is made to receive an optical projection of an image.

45. The apparatus according to claim 40, wherein said surface for generating said displayed image thereon is curved.

46. The apparatus according to claim 41, wherein the number of said plurality of image elements does not exceed the number of cones on said part of the retina.

47. The apparatus according to claim 41, wherein the number of said plurality of image elements does not exceed the number of optical fibers on said part of the retina.

48. The apparatus according to claim 40, wherein said housing is cranially mountable.

49. The apparatus according to claim 40, wherein:
said surfaces
said optical element,
are provided for each eye,
for establishing an optical communication between a displayed image formed on each one of said surfaces and each retina.

50. The apparatus according to claim 49, wherein:
two of said surfaces are provided,
a different displayed image is generated on each one of said surfaces, to stimulate a stereoscopic perception by said viewer.

51. The apparatus according to claim 40 including a light source, wherein said surface is disposed on said optical path between said light source and said eye's retina to transmit light from said light source towards said selectable part of the retina.

52. The apparatus according to claim 51, wherein light reaches the retina intermittently.

53. The apparatus according to claim 52, wherein said light source emits light at a constant frequency of at least 13 Hz.

54. The apparatus according to claim 40, wherein each one of said two displayed images is generated at a constant frequency of at least 13 Hz.

55. The apparatus according to claim 54, wherein said light path is obstructed intermittently.
